(19) United States
(12) Patent Application Publication
(10) Pub. No.: US 2026/0226516 A1
(43) Pub. Date: Aug. 6, 2026

(54) PHOSPHOLIPID-BASED SURFACTANT USING SKIN FLORA AND MANUFACTURING METHOD THEREOF

(71) Applicants: COSMAX AB Inc., Seoul (KR); COSMAX, INC., Gyeonggi-do (KR)

(72) Inventors: Ju Yun KIM, Seoul (KR); Ju Hyun SON, Incheon (KR); Kwang Nyeon KIM, Gyeonggi-do (KR)

(21) Appl. No.: 19/151,492

(22) PCT Filed: May 20, 2024
(86) PCT No.: PCT/KR2024/006781

§ 371 Date: Jul. 28, 2025

(30) Foreign Application Priority Data
May 31, 2023 (KR) 10-2023-0070150

(51) Int. Cl. C12P 7/6481; A61K 8/55; A61Q 19/00

(52) U.S. Cl. CPC C12P 7/6481; A61K 8/553; A61Q 19/00; A61K 2800/85

(57) ABSTRACT

The present invention relates to a phospholipid-based surfactant prepared in an environmentally friendly and human-friendly manner using skin flora, and a preparation method therefor. The present invention can provide an environmentally friendly and human-friendly composition in substitution for a synthetic surfactant. In addition, the surfactant of the present invention can be variously applied to the fields of food, detergents, cosmetics, and the like.

### TECHNICAL FIELD

The present invention relates to a phospholipid-based surfactant prepared in an environmentally friendly and human-friendly manner using skin flora, and a method for preparing the same. This application claims the benefit of priority from Korean Patent Application No. 10-2023-0070150, filed on May 31, 2023, the entire content of which is incorporated herein by reference.

### BACKGROUND ART

The human body can be a habitat for various microorganisms, and microorganisms form a symbiotic relationship with the host and affect the host. Among these, skin flora present on the skin are microorganisms that live on the surface of the skin and are mainly found in the outermost layer of the epidermis and the upper part (upper layer) of the hair follicles. There are approximately 1,000 species of microorganisms as the skin flora. Among the microorganisms present on the skin, aerobic microorganisms can secrete lipolytic enzymes that can utilize lipids in the epidermal layer.

Phospholipids are one of the basic components of cell membranes and can be found naturally in animals, plants, or microorganisms. In general, phospholipids in cell membranes can be extracted by disrupting cells. Since chemical and physical disruption is applied during this process, it is difficult to view phospholipids obtained in this way as environmentally friendly and human-friendly. For Gram-positive aerobic bacteria, the composition of extracellular phospholipids has been studied, but this was analyzed by disrupting extracellular vesicles contained in biofilms cultured on the surface, and no studies have been conducted on extracellular phospholipids cultured and obtained in a liquid phase without disrupting cells.

In general, phospholipids are used as emulsifiers, and phospholipid-based functional ingredients are called lecithins. The phospholipids found in commercially available lecithin mainly include phosphatidylcholine (PC), phosphatidylethanolamine (PE), phosphatidylinositol (PI), and phosphatidic acid (PA).

In recent years, demand for environmentally friendly and human-friendly products has been increasing in fields such as foods, detergents, and cosmetics. Accordingly, research is needed to replace synthetic surfactants with environmentally friendly and human-friendly surfactants.

### DETAILED DESCRIPTION OF THE INVENTION

### Technical Problem

The purpose of the present invention is to provide an environmentally friendly and human-friendly phospholipid-based surfactant by using skin flora. This application claims the benefit of priority from Korean Patent Application No. 10-2023-0070150, filed on May 31, 2023, the entire content of which is incorporated herein by reference.

### Solution to Problem

In order to solve the above problem, the present invention provides a phospholipid-based surfactant prepared from a microorganism of skin flora with the accession number KCTC15741BP (Korea Research Institute of Bioscience and Biotechnology, Biological Resource Center).

In one embodiment, the surfactant may comprise a phospholipid with or without amine or sugar groups bound to. Specifically, the surfactant may include one or more of phosphatidyl glycerol, ceramide phosphoryl ethanolamine, phosphatidyl ethanolamine, lysophosphatidyl ethanolamine, and branched fatty acid ester of hydroxy fatty acid.

According to another embodiment of the present invention, there is provided a method for preparing a phospholipid-based surfactant, comprising:
a seed culture step of culturing a strain of skin flora with the accession number KCTC15741BP (Korea Research Institute of Bioscience and Biotechnology, Biological Resource Center) to prepare a seed culture;
a pre-culture step of culturing the seed culture in a medium containing vegetable oil to prepare a pre-culture;
a main fermentation step of adding the pre-culture to a medium containing vegetable oil to prepare a main fermented product;
a step of separating a precipitate from the fermented product; and
a step of extracting phospholipids from the precipitate.

According to one embodiment, the medium in the seed culture step may contain casein, soybean, dextrose, sodium chloride and dipotassium phosphate. Specifically, the medium in the seed culture step may contain 5 to 30 g/L of casein, 1 to 10 g/L of soybean, 1 to 6 g/L of dextrose, 1 to 10 g/L of sodium chloride, and 1 to 6 g/L of dipotassium phosphate.

According to one embodiment, the medium in the pre-culture step may contain soybean, yeast extract, glycerin, potassium phosphate, dipotassium phosphate and vegetable oil. Specifically, the medium in the pre-culture step may contain 0.01 to 3 g/L of soybean, 0.1 to 5 g/L of yeast extract, 0.1 to 5 g/L of glycerin, 1 to 15 g/L of potassium phosphate, 1 to 10 g/L of dipotassium phosphate, and 10 to 100 ml/L of vegetable oil.

According to one embodiment, the medium in the main fermentation step may contain soybean, yeast extract, glycerin, potassium phosphate, dipotassium phosphate, ammonium sulfate, magnesium sulfate, potassium nitrate, calcium chloride, and vegetable oil. Specifically, the medium in the main fermentation step may contain 0.01 to 3 g/L of soybean, 0.1 to 5 g/L of yeast extract, 0.01 to 5 g/L of glycerin, 1 to 10 g/L of potassium phosphate, 1 to 10 g/L of dipotassium phosphate, 0.1 to 5 g/L of ammonium sulfate, 0.01 to 5 g/L of magnesium sulfate, 0.01 to 3 g/L of potassium nitrate, 0.01 to 3 g/L of calcium chloride and 10 to 800 ml/L of vegetable oil.

According to one embodiment, the seed culture step may comprise culturing under aerobic conditions of 25 to 35° C., the pre-culture step may comprise introducing 10 to 500 g/L of the seed culture and culturing under aerobic conditions of 25 to 35° C., and the main fermentation step may comprise introducing 10 to 500 g/L of the pre-culture and culturing under aerobic conditions of 25 to 35° C.

According to one embodiment, the method may comprise a step of subculturing to the pre-culture medium when the absorbance at a wavelength of 600 nm is 0.05 to 0.2 in the seed culture step;
a step of subculturing to the main fermentation when the absorbance at a wavelength of 600 nm is 0.2 to 1 in the pre-culture step; or
a step of completing the main fermentation at 60 to 150 hours after the start of the main fermentation.

According to one embodiment, the step of separating the precipitate may comprise centrifuging the fermented product to recover the precipitate.

According to one embodiment, the step of extracting the phospholipids may comprise mixing the recovered precipitate and a solvent to obtain a filtrate, and concentrating the filtrate.

Specific details of other implementations according to the present invention are included in the detailed description below.

### Effect of the Invention

The phospholipid-based surfactant using skin flora according to the present invention can replace a synthetic surfactant. The surfactant of the present invention can be applied to fields such as foods, detergents, and cosmetics to provide environmentally friendly and human-friendly products.

### BRIEF DESCRIPTION OF THE DRAWINGS

FIG. 1 is a photograph of a filtrate obtained according to Example.

FIG. 2 is a photograph of a phospholipid-based surfactant recovered according to Example.

FIG. 3 is a chromatogram showing the results of TLC analysis for the phospholipid-based surfactant of Example.

FIG. 4 is a chromatogram showing the results of TLC analysis according to the fermentation time.

FIG. 5 is a photograph visually observing the difference in emulsions depending on the presence or absence of phospholipids of Example.

### BEST MODE FOR CARRYING OUT THE INVENTION

The present invention can be modified in various ways and has many embodiments, and specific embodiments will be exemplified and described in detail. However, this is not intended to limit the present invention to specific embodiments, but should be understood to include all transformations, equivalents, or substitutes included in the spirit and technical scope of the present invention. In explaining the present invention, if it is determined that a detailed description of a related known technology may obscure the gist of the present invention, the detailed description is omitted.

Hereinafter, a phospholipid-based surfactant using skin flora according to the present invention and a method for producing the same will be described in detail.

The phospholipid-based surfactant of the present invention can be produced by microorganisms, for example, skin flora. Specifically, the present invention may utilize a strain of Epidermidibacterium keratini sp., specifically, a strain having the accession number KCTC15741BP (Korea Research Institute of Bioscience and Biotechnology, Biological Resource Center).

Skin flora may produce phospholipid-based surfactant components when cultured and fermented under specific conditions. Phospholipid-based surfactant components are environmentally friendly and human-friendly, so they can solve various shortcomings and problems of synthetic surfactants.

According to one aspect, the present invention provides a phospholipid-based surfactant produced by a microorganism of skin flora with the accession number KCTC15741BP (Korea Research Institute of Bioscience and Biotechnology, Biological Resource Center). The surfactant of the present invention may comprise a phospholipid with or without amine or sugar groups bound to. For example, it may include glycerophospholipid, sphingolipid, glycerophosphoethanolamine, fatty acid ester, sterol, sphingoid base, etc. Specifically, the surfactant of the present invention may include one or more of phosphatidyl glycerol (PG), ceramide phosphoryl ethanolamine (sphingomyelin, Cer-PE), phosphatidyl ethanolamine (PE), lysophosphatidyl ethanolamine (LPE), and a fatty acid derivative such as branched fatty acid ester of hydroxy fatty acid (FAHFA).

According to another embodiment of the present invention, there is provided a method for preparing a phospholipid-based surfactant, comprising a seed culture step of culturing a strain of skin flora with the accession number KCTC15741BP (Korea Research Institute of Bioscience and Biotechnology, Biological Resource Center) to prepare a seed culture; a pre-culture step of culturing the seed culture in a medium containing vegetable oil to prepare a pre-culture; a main fermentation step of adding the pre-culture to a medium containing vegetable oil to prepare a main fermented product; a step of separating a precipitate from the fermented product; and a step of extracting phospholipids from the precipitate.

The vegetable oil used in the present invention is not particularly limited as long as it is a vegetable oil without cytotoxicity. Specifically, for example, the vegetable oil may include, but is not particularly limited to, one or more of edible or human-friendly oils such as macadamia oil, sunflower seed oil, grape seed oil, canola oil, rice germ oil, olive oil, soybean oil, argan oil, brown rice oil, perilla oil, sesame oil, almond oil, peanut oil, corn oil, red ginseng oil, avocado oil, coconut oil, rosehip oil, vitamin tree seed oil, shea tree fruit oil, oil palm oil, bergamot fruit oil, camellia seed oil, safflower seed oil, apricot kernel oil, poppy seed oil, evening primrose seed oil, castor seed oil, green tea seed oil, meadowfoam seed oil, flax seed oil, and hemp seed oil.

According to one embodiment, the medium in the seed culture step may contain casein, soybean, dextrose, sodium chloride and dipotassium phosphate. Specifically, the medium in the seed culture step may contain 5 to 30 g/L, for example, 10 to 25 g/L, or 15 to 20 g/L of casein, 1 to 10 g/L, for example, 2 to 8 g/L, or 2 to 4 g/L of soybean, 1 to 6 g/L, for example, 1 to 4 g/L, or 2 to 3 g/L of dextrose, 1 to 10 g/L, for example 2 to 8 g/L, or 4 to 6 g/L of sodium chloride, and 1 to 6 g/L, for example 2 to 5 g/L, or 2 to 3 g/L of dipotassium phosphate.

According to one embodiment, the medium in the pre-culture step may contain soybean, yeast extract, glycerin, potassium phosphate, dipotassium phosphate and vegetable oil. Specifically, for example, the medium in the pre-culture step may contain 0.01 to 3 g/L, for example 0.05 to 2 g/L, or 0.05 to 1 g/L of soybean, 0.1 to 5 g/L, for example 0.1 to 3 g/L, or 0.5 to 2 g/L of yeast extract, 0.1 to 5 g/L, for example 0.1 to 3 g/L, or 0.5 to 2 g/L of glycerin, 1 to 15 g/L, for example 1 to 10 g/L, or 3 to 6 g/L of potassium phosphate, 1 to 10 g/L, for example 1 to 8 g/L, or 2 to 6 g/L of dipotassium phosphate, and 10 to 100 ml/L, for example 20 to 80 ml/L, or 30 to 60 ml/L of vegetable oil.

According to one embodiment, the medium in the main fermentation step may contain soybean, yeast extract, glycerin, potassium phosphate, dipotassium phosphate, ammonium sulfate, magnesium sulfate, potassium nitrate, calcium chloride, and vegetable oil. Specifically, for example, the medium in the main fermentation step may contain 0.01 to 3 g/L, for example 0.05 to 2 g/L, or 0.05 or 1 g/L of soybean, 0.1 to 5 g/L, for example 0.1 to 3 g/L, or 0.5 to 2 g/L of yeast extract, 0.01 to 5 g/L, for example 0.05 to 3 g/L, or 0.05 to 2 g/L of glycerin, 1 to 10 g/L, for example 1 to 10 g/L, or 3 to 6 g/L of potassium phosphate, 1 to 10 g/L, for example 1 to 8 g/L, or 2 to 6 g/L of dipotassium phosphate, 0.1 to 5 g/L, for example 0.5 to 3 g/L, or 0.5 to 2 g/L of ammonium sulfate, 0.01 to 5 g/L, for example 0.05 to 3 g/L, or 0.05 to 1 g/L of magnesium sulfate, 0.01 to 3 g/L, for example 0.05 to 2 g/L, or 0.05 to 1 g/L of potassium nitrate, 0.01 to 3 g/L, for example 0.05 to 2 g/L, or 0.1 to 1 g/L of calcium chloride, and 10 to 800 ml/L, for example 50 to 500 ml/L, or 100 to 300 ml/L, or 150 to 250 ml/L of vegetable oil.

According to one embodiment, the seed culture step of the present invention may comprise culturing under aerobic conditions of 25 to 35° C., for example 28 to 32° C. In the seed culture step of the present invention, the seed culture may be diluted in a sterilized solution of 0.85% sodium chloride to 1 to 10% (v/v), for example 3 to 8% (v/v), and the seed culture may be subcultured to the pre-culture when the absorbance is 0.05 to 0.2, for example, 0.05 to 0.15 as measured at a wavelength of 600 nm using a spectrophotometer.

According to one embodiment, the pre-culture step of the present invention may comprise adding 10 to 500 g/L, for example 50 to 300 g/L, or 50 to 200 g/L of the seed culture, and culturing under aerobic conditions at 25 to 35° C., for example 28 to 32° C. Here, the ventilation rate can be set to 10 to 50 NL/min, for example, 20 to 40 NL/min, and the stirring speed may be set to 50 to 300 rpm, 100 to 200 rpm, or 150 to 200 rpm, for example. In the pre-culture step of the present invention, the pre-culture may be diluted in a sterilized solution of 0.85% sodium chloride to 1 to 10% (v/v), for example 3 to 8% (v/v), and the pre-culture may be subcultured to the main fermentation when the absorbance is 0.2 to 1, for example, 0.3 to 0.8 as measured at a wavelength of 600 nm using a spectrophotometer.

According to one embodiment, the main fermentation step of the present invention may comprise adding 10 to 500 g/L of pre-culture, for example 50 to 300 g/L, or 50 to 200 g/L, and culturing under aerobic conditions at 25 to 35° C., for example 28 to 32° C. Here, the ventilation rate can be set to 100 to 500 NL/min or 200 to 400 NL/min, for example, and the stirring speed may be set to 300 to 900 rpm, 400 to 800 rpm, or 500 to 700 rpm, for example. The main fermentation step of the present invention may be carried out for, for example, 60 to 150 hours, 100 to 150 hours, or up to 144 hours from the start of the main fermentation.

According to one embodiment, the step of separating the precipitate of the present invention may comprise centrifuging the fermented product to recover the precipitate. The step of separating the precipitate of the present invention may comprise centrifuging at 3,000 to 10,000 rpm, 4,000 to 9,000 rpm, or 5,000 to 8,000 rpm, for example. Since the phospholipids of the present invention are found outside of cells without disruption of cells, precipitates containing both bacterial cells and extracellular phospholipids are recovered.

According to one embodiment, the step of extracting the phospholipids of the present invention may comprise mixing the recovered precipitate and a solvent to obtain a filtrate, and concentrating the filtrate. Specifically, the step of extracting the phospholipids of the present invention may comprise adding a solvent 2 to 20 times, for example 5 to 15 times of the weight of the recovered precipitate, and stirring at 25 to 35° C., or 28 to 32° C., for example, at 100 to 200 rpm or 130 to 180 rpm, for example, for 10 to 30 hours, 15 to 25 hours, or 15 to 20 hours, for example. Examples of the solvent that can be used include water, ethanol, methanol, butyleneglycol, pentyleneglycol, dipropylene glycol, propandiol, hexanediol, butanol, ethyl acetate, and the like, and specifically, 95% (v/v) ethanol can be used, for example.

According to one embodiment, the phospholipid-based surfactant of the present invention can be used without limitation in various fields where surfactants are applied, such as foods, detergents, and cosmetics.

According to one embodiment, the phospholipid-based surfactant of the present invention may also be used together with natural surfactants such as proteins, polysaccharides, saponins, cocobetaine, xanthan gum, and beeswax.

When the surfactant of the present invention is used as an additive in the food field, it may replace some or all of gelatin, fatty acid ester, carrageenan, propylene glycol, triacetin, calcium stearyl lactate, lecithin, sodium dodecyl sulfate, and the like, but is not limited to the above types.

When the surfactant of the present invention is used as a surfactant for detergents, it may replace some or all of alkylbenzene sulfonate, alpha-olefin sulfonate, alkyl sulfate ester salt, polyoxyethylene alkyl ether sulfate, alkane sulfonate, alpha-sulfo fatty acid ester salt, dialkyldimethyl ammonium salt, imidazolium salt, alkyldimethyl benzyl ammonium salt, polyoxyethylene alkyl ether, alkyl dimethyl amine oxide, fatty acid alkanol amide, alkyl polyglucoside, alkyl phenol ethoxylate, alkyl betaine, alkyl sulfobetaine, and the like, but is not limited to the above types.

When the surfactant of the present invention is used as a surfactant for cosmetics, it may replace some or all of alkylbenzene sulfonate, alcohol ethoxylate, alcohol ethyl oxide, alcohol ether sulfonate, acyl glutamate, alkylamine oxide, alpha olefin sulfonate, alkylphenol ethoxylate, alkyl polyglycoside, alkyl polypentoside, alcohol sulfonate, alkyl sulfobetaine, dicetyl ether sulfonate, ethylene oxide/propylene oxide block copolymer, fatty acid alkanol amide, fatty alcohol ether sulfate, fatty amine oxide, fatty alcohol sulfate, linear alkylbenzene, linear alkylbenzene sulfonate, methyl ester ethoxylate, and the like, but is not limited to the above types.

Hereinafter, embodiments of the present invention will be described in detail so that a person skilled in the art can easily implement the present invention. However, the present invention may be implemented in various different forms and is not limited to the embodiments described herein.

### Example: Manufacturing of Phospholipid-Based Surfactant Using Skin Flora

### Seed Culture

A medium was prepared containing 17.0 g/L of casein (pancreatic digest of casein), 3.0 g/L of soybean (papain digest of soybean), 2.5 g/L of dextrose, 5.0 g/L of sodium chloride, 2.5 g/L of dipotassium phosphate, and purified water to make a total medium volume of 1 L.

The microbiome with the accession number KCTC15741BP (Korea Research Institute of Bioscience and Biotechnology, Biological Resource Center) was inoculated into the prepared medium, the medium was cultured under aerobic conditions 30° C. to prepare a seed culture.

The seed culture was diluted in a sterilized solution of 0.85% (w/v) sodium chloride to 5% (v/v) and, the seed culture was subcultured to the pre-culture when the absorbance is 0.1 as measured at a wavelength of 600 nm using a spectrophotometer (BioTek, EPOCH2C).

### Pre-Culture

A medium was prepared containing 0.1 g/L of soybean (papain digest soybean), 1.0 g/L of yeast extract, 1.0 g/L of glycerin, 4.5 g/L of monopotassium phosphate, 3.0 g/L of dipotassium phosphate, 50 ml/L of olive oil, and water to make a total medium volume of 1 L. However, olive oil was added in a volume unit.

100 g/L of the seed culture was added to the prepared medium and cultured at a ventilation rate of 30 NL/min, a culture temperature of 30° C., and a stirring speed of 180 rpm to prepare a pre-culture.

The pre-culture was diluted in a sterilized solution of 0.85% (w/v) sodium chloride to 5% (v/v), and the pre-culture was subcultured when the absorbance is 0.5 to 0.6 as measured at a wavelength of 600 nm using a spectrophotometer.

### Main Fermentation

A medium was prepared containing 0.1 g/L of soybean (papain digest soybean), 1.0 g/L of yeast extract, 0.1 g/L of glycerin, 4.5 g/L of monopotassium phosphate, 3.0 g/L of dipotassium phosphate, 1.0 g/L of ammonium sulfate, 0.5 g/L of magnesium sulfate heptahydrate, 0.1 g/L of potassium nitrate, 0.3 g/L of calcium chloride, 200 ml/L of olive oil, and water to make a total medium volume of 1 L. However, olive oil was added in a volume unit.

100 g/L of the pre-culture was added to the prepared medium and cultured at a ventilation rate of 300 NL/min, a culture temperature of 30° C., and a stirring speed of 600 rpm to prepare a main fermented product. The main fermentation was carried out for 144 hours.

### Extraction

The main fermented product was centrifuged at 7,000 rpm using a high-speed continuous centrifuge (DHC, SM-400) to recover the precipitate.

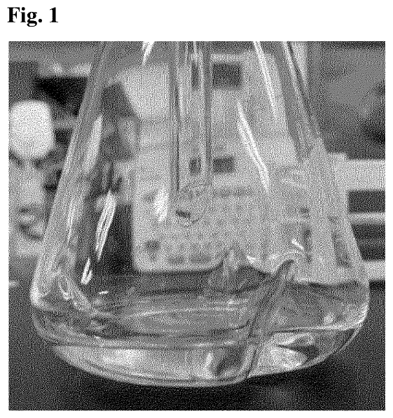

95% ethanol equivalent to 10 times the weight of the recovered precipitate was added and stirred at 30° C. and 150 rpm for 18 hours. Afterwards, the solution was filtered through a glass fiber filter having a pore size of 0.45 m to obtain a yellow transparent filtrate. A photograph for visually observing the obtained filtrate is shown in FIG. 1.

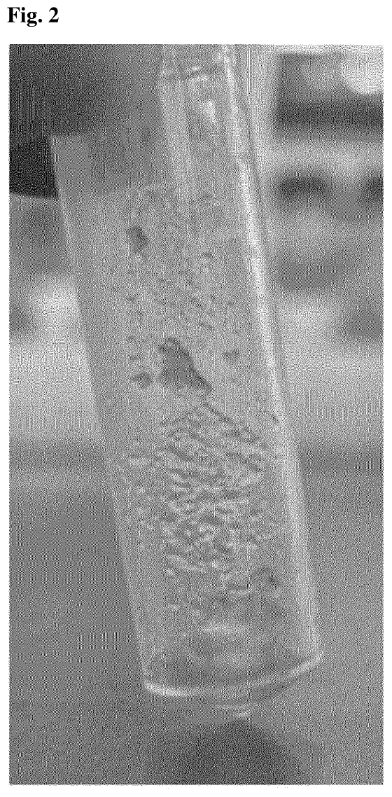

The filtrate was concentrated under reduced pressure to recover extracellular phospholipids as a yellow solid, and a photograph for visually observing the recovered phospholipids is shown in FIG. 2. The yield of the main fermented product after 114 hours of main fermentation was approximately 5% (w/w).

### Comparative Example 1

The seed culture, pre-culture and main culture were performed in the same manner as in Example, except that Bacillus subtilis (accession number KCCM10619) was used.

### Comparative Example 2

The seed culture, pre-culture and main culture were performed in the same manner as in Example, except that Staphylococcus aureus (accession number KCTC3881) was used.

### Experimental Example 1: Comparison of Extracellular Phospholipid Production According to the Type of Skin Flora

The amounts of extracellular phospholipid production of different types of skin flora and the strain with the accession number KCTC15741BP (Korea Research Institute of Bioscience and Biotechnology, Biological Resource Center) according to Example were compared. Gram-positive aerobic bacteria were selected as different skin flora.

The extent of extracellular phospholipid production was measured as follows. First, the seeds of Comparative Example 1, Comparative Example 2, and Example were seed cultured and pre-cultured, respectively, and the fermented solution which was fermented for 144 hours was centrifuged at 13,000 rpm for 3 minutes. Then, the supernatant was removed to obtain the precipitate. Each precipitate of the same weight was extracted using the solvent extraction method of Example, and concentrated under reduced pressure to determine the yield of the concentrate.

The results are shown in Table 1.

TABLE 1

| | Comparative | Comparative | |
|---|---|---|---|
| Item | Example 1 | Example 2 | Example |
| Yield (%_{w/w}) | 0.37 | 0.12 | 5.03 |

In Comparative Example 2, there was almost no increase in precipitates and extracellular phospholipids were also hardly observed, compared to Example. Comparative Example 1 showed an increase in precipitates over time, but a significantly low yield of extracellular phospholipids. In contrast, Example showed the yield of extracellular phospholipids of approximately 5% (w/w).

A phospholipid is a basic component of cell membranes and can be extracted from all cells having cell membranes. However, phospholipids that make up cell membranes are hardly to obtain without chemical and physical disruption of the cells.

In the above Experimental Example, phospholipids existing outside the cells were extracted using only 95% ethanol without disrupting the cells. In the case of Comparative Example 2, the yield of phospholipids was observed to be low even though the amount of precipitates increased noticeably with the incubation time, whereas Example showed a high yield of phospholipids. It showed that, unlike Comparative Example 2, the precipitates of the microbiome with the accession number KCTC15741BP (Korea Research Institute of Bioscience and Biotechnology, Biological Resource Center) according to Example contain not only bacterial cells but also phospholipids existing outside the cell.

### Experimental Example 2: TLC (Thin Layer Chromatography) Analysis

The extracellular phospholipids obtained according to Example were dissolved in chloroform. The solution was spotted onto a silica gel plate (TLC Silica gel 60 F254, Merck; 10 cm×5 cm) at the starting point (base) and developed with a mobile phase containing chloroform, methanol, and distilled water (6.5:2.5:0.4, v:v:v) to the end point (front).

The components spread on the naturally dried TLC plate were individually stained with different reagents to develop color. Lipid components were color developed using Nile red reagent. The reagent was prepared at a concentration of 0.001 g/L in a solvent containing methanol and distilled water in 8:2 ratio. The prepared reagent was sprayed on a naturally dried TLC plate and completely dried, and then it was immersed in a 0.0004% (w/w) solution of sodium hypochlorite to decolorize any unnecessarily stained portion. After the decolorized TLC plate was completely dried, ultraviolet light was irradiated at a wavelength of 365 nm to observe the red-stained spots.

For ninhydrin, the reagent was prepared by mixing it in ethanol at a concentration of 5 g/L. The prepared reagent was sprayed onto a naturally dried TLC plate, dried, and heated at 100° C. for 5 minutes to develop the component bound to the amine group into a purple spot.

For molybdenum blue, after spraying it on a naturally dried TLC plate, it was left at room temperature and in the dark for 5 minutes to develop the component bound to the phosphate group into a blue spot.

For orcinol, the reagent was prepared at a concentration of 2 g/L in 20% sulfuric acid. The prepared reagent was sprayed onto a naturally dried TLC plate, dried, and heated at 100° C. for 5 minutes to develop the sugar component into a purple spot.

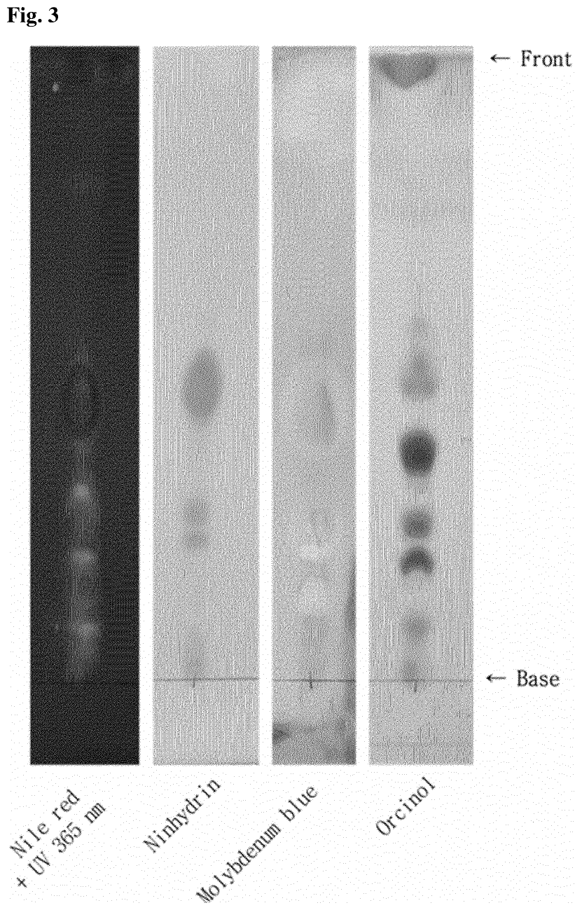

The TLC results are shown in FIG. 3. As a result of TLC development and reagent color development, it was observed that the extract according to Example contained phospholipids, phospholipids bound to amine groups, and phospholipids bound to sugar residues.

### Experimental Example 3: TLC Pattern Changes According to Incubation Time

In order to observe the change in phospholipid composition according to the fermentation time, the seed culture and pre-culture stages of Example were performed, followed by the main fermentation. Herein, precipitates were obtained from each sample at 36 hours, 72 hours, and 144 hours after the start of fermentation, respectively. Each precipitate of the same weight was extracted using the solvent extraction method of Example to obtain extracellular phospholipids for each time period.

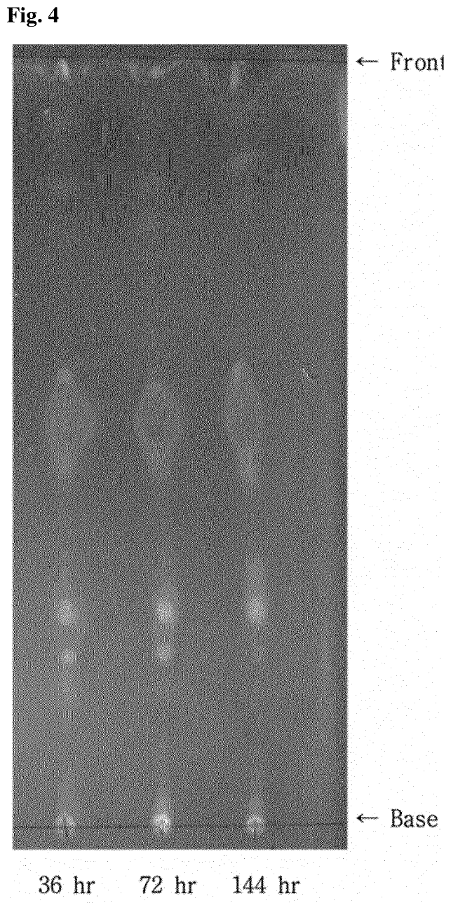

The obtained extracellular phospholipids were developed by TLC with Nile red reagent in the same manner as in Experimental Example 2. The results are shown in FIG. 4.

As shown in FIG. 4, the phospholipids obtained from the precipitates did not show any qualitative difference according to the incubation time. It is thought that there will be a quantitative difference according to precipitate weight rather than a qualitative difference.

### Experimental Example 4: HPLC TOF-MS Analysis

Extracellular phospholipids obtained from the precipitate taken 144 hours after the start of main fermentation were dissolved in tetrahydrofuran, and diluted in methanol, and analyzed using a high-resolution mass spectrometer. The instrument used was a high-resolution mass spectrometer (AB Sciex, Triple TOF 5600+) connected to HPLC (thermo scientifinc, Ultimate3000). The analysis conditions are as shown in Tables 2 and 3.

TABLE 2

| Column | Waters BEH C18 (2.1 mm × 100 mm, 1.7 um) | | |
|---|---|---|---|
| Column | 45° C. | | |
| Temperature | | | |
| Injection volume | 0.4 ul | | |
| Mobile phase | A = 0.1% acetic acid, 10 mM ammonium formate | | |
| | in acetonitrile/water (6:4) | | |
| | B = 0.1% acetic acid, 10 mM ammonium formate | | |
| | in acetonitrile/Isopropyl alcohol (1:9) | | |
| Gradient | Time (min) | A (%) | B (%) |
| | 0 | 95 | 5 |
| | 0.5 | 95 | 5 |
| | 20 | 0 | 100 |
| | 25 | 0 | 100 |
| | 25.5 | 95 | 5 |
| | 30 | 95 | 5 |

| | |
|---|---|
| Flow rate | 0.4 ml/min |

TABLE 3

| Ionization source | Electrospray ionization (ESI) | |
|---|---|---|
| Ionization mode | Positive and negative | |
| MS scan range | 100~2000 | m/z |
| MS/MS scan range | 30~2000 | m/z |
| Gas Temperature | 500° | C. |
| Ionspray voltage | Positive: 5.5 kV, Negative: 4.5 kV | |
| Nebulizing gas | 50 | psi |
| Heating gas | 50 | psi |

| | |
|---|---|
| Collision gas | N₂ |
| Collision Energy | POS: 35 ± 15, Neg: −35 ± 15 |
| Collision Energy spread | 15 |

The components were analyzed in positive mode and negative mode, and the components were predicted based on the lipid library program, as shown in Table 4.

TABLE 4

| No. | Type | constituent | INCHIKEY |
|---|---|---|---|
| 1 | Glycerophospholipids | PG 16:0 18:1 | PAZGBAOHGQRCBP- |
| | | | ZCXUNETKNA-N |
| 2 | Phosphosphingolipids | PE-Cer | WEURQMDBJOJBNR- |
| | | 25:1; 20/26:2; 0 | SMJXLIDUNA-N |
| 3 | Glycerophospho- | PE 34:2 | RAMNOXDFBNFSFC- |
| | ethanolamines | | LJSPAGPLSA-N |
| 4 | | PE 34:1 | FHQVHHIBKUMWTI- |
| | | | ZCXUNETKSA-N |
| 5 | | PE 16:0 16:1 | CZOSTDZGCCEZTJ- |
| | | | PEZBUJJGNA-N |
| 6 | | LPE 16:0 | YVYMBNSKXOXSKW- |
| | | | UHFFFAOYSA-N |
| 7 | | LPE 14:0 | RPXHXZNGZBHSMJ- |
| | | | UHFFFAOYSA-N |
| 8 | Fatty esters | FAHFA 43:4 | UHGXBKJKWOYPQH- |
| | | | CSJOWCBCSA-N |
| 9 | | FAHFA 43:2 | VWUMNFKMUWUSPE- |
| | | | XOMYGGGSSA-N |
| 10 | | FAHFA 41:3 | LNRJZDOCBHAVAF- |
| | | | XGDIZPIPSA-N |
| 11 | | FAHFA 41:2 | XGDLBOHEFZAITF- |
| | | | XINOORGGSA-N |
| 12 | | FAHFA 40:0 | SCKNNTGGHWOXSY- |
| | | | UHFFFAOYSA-N |
| 13 | | FAHFA 39:3 | PZFOSNSZMAGVLN- |
| | | | PJKXCORCSA-N |
| 14 | | FAHFA 38:0 | GYOQIEBLVVLMJZ- |
| | | | UHFFFAOYSA-N |
| 15 | | FAHFA 36:2 | DHOLNZBDURYVOE- |
| | | | SVADLMEDSA-N |
| 16 | | FAHFA 36:1 | PGKKGBQMNNEIHV- |
| | | | PFONDFGASA-N |
| 17 | | FAHFA 17:2/26:0 | VWUMNFKMUWUSPE- |
| | | | XOMYGGGSNA-N |
| 18 | Sterols | ST 24:1; 04/19:1 | IQXMCKPTJXIOSC- |
| | | | YPKPFQOONA-N |
| 19 | Sphingoid bases | SPB 22:1; 20 | BSHKJMWKIAPLFB- |
| | | | VHEBQXMUNA-N |
| 20 | | SPB 20:1; 20 | HTJSZHKGNMXZJN- |
| | | | WUKNDPDINA-N |
| 21 | Diacylglycerols | DG 0-19:1 17:2 | PZYGHOOOWOZPPW- |
| | | | BLLZRTNRNA-N |
| 22 | | DG 18:1 20:1 | AKCNIDZVBDSVNW- |
| | | | HWSICPKZNA-N |
| 23 | Triacylglycerols | TAG 54:4 | UNWVBURSXHYQJM- |
| | | | KZCPXVGCSA-N |
| 24 | | TAG 46:3 | QXDIHCMYHHVYQI- |
| | | | AAPVPQQHSA-N |
| 25 | | TG 14:0 16:1 18:4 | YURZMLNQYDXMPP- |
| | | | DLIYJMOJNA-N |

Results with a mass accuracy score and MS score of 0.8 to 1.00 were selected on the lipid library program. As a result of component analysis, phospholipids such as phosphatidyl glycerol (PG), ceramide phosphoryl ethanolamine (sphingomyelin, Cer-PE), phosphatidyl ethanolamine (PE), lysophosphatidyl ethanolamine (LPE), and a fatty acid derivative such as branched fatty acid ester of hydroxy fatty acid (FAHFA) were detected, except for triacylglycerols and diacylglycerols.

### Experimental Example 5: Emulsification Index Test

To confirm whether the extracellular phospholipids of FIG. 2 are applicable to the formulation and exhibit surfactant properties as a bipolar substance, the emulsification stability index was measured.

First, among five solvents applicable to cosmetics, a solvent in which extracellular phospholipids according to Example are soluble was selected. The solvents used herein were 1,3-butyleneglycol (1,3-BG), pentyleneglycol (PG), dipropylene glycol (DPG), 1,3-propandiol (PDO), and 1,2-hexanediol (1,2-HXD).

As a result of dissolving in each solvent, the extracellular phospholipids according to Example showed solubility only in pentylene glycol.

Next, the emulsification stability index of extracellular phospholipids according to Example was measured. The experimental group was prepared by putting 1 ml of a solution containing 0.1% (w/v) of extracellular phospholipids according to Example in pentylene glycol, 10 ml of purified water, and 10 ml of MCT oil (WOOSUNG CNT, Ltd/Radia7104) into a 50 ml conical tube and then vortexing vigorously for 3 minutes. A control group was prepared in the same manner as the experimental group, except that the composition was used excluding extracellular phospholipids according to Example. That is, the control group was prepared by introducing 1 ml of pentylene glycol, 10 ml of purified water, and 10 ml of MCT oil (WOOSUNG CNT, Ltd/Radia7104) into a 50 ml conical tube and then vortexing vigorously for 3 minutes.

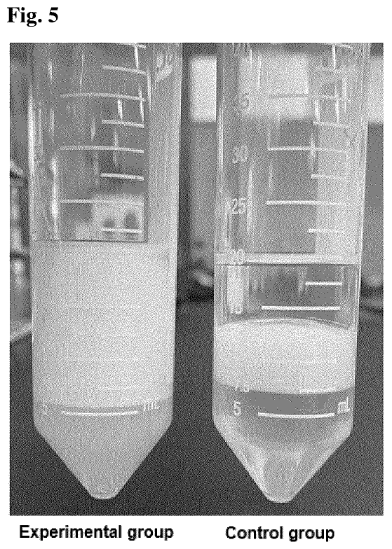

After each mixture of the experimental group and control group was allowed to stand in a 25° C. chamber for 24 hours, the height of the emulsified layer was measured. The appearance after 24 hours of standing is shown in FIG. 5. Equation 1 below was used to calculate the emulsification stability index.

$$[ Equation 1 ]$$

$$Emulsification stability index = ( height of emulsion layer ) / ( total height of mixture )$$

To confirm the functional properties of extracellular phospholipids, 1 ml of pentylene glycol, 10 ml of purified water, and 10 ml of MCT oil (WOOSUNG CNT, Ltd/Radia7104) were mixed and allowed to stand in the same manner and used as a control group.

As a result of the experiment, the emulsification stability index of the experimental group was 0.54, and that of the control group was 0.22, so it was confirmed that the emulsification stability index was approximately 2.45 times higher when the extracellular phospholipids according to Example were included. Therefore, it can be seen that the extracellular phospholipids obtained according to Example exhibit surfactant properties.

As described above, the phospholipid-based surfactant of the present invention is produced by skin flora, is human-friendly, can minimize irritation to the human body and the environment, and includes a phospholipid component having functional properties as a surfactant.

While specific embodiments of the present invention have been described in detail above, it should be understood by those skilled in the art that such specific description is merely a preferred embodiment, and the scope of the present invention is not limited to the specific embodiments described above.

1. A phospholipid-based surfactant prepared from a microorganism of skin flora with the accession number KCTC15741BP (Korea Research Institute of Bioscience and Biotechnology, Biological Resource Center).

2. The phospholipid-based surfactant according to claim 1, wherein the surfactant comprises a phospholipid with or without amine or sugar groups bound to.

3. The phospholipid-based surfactant according to claim 1, wherein the surfactant includes one or more of phosphatidyl glycerol, ceramide phosphoryl ethanolamine, phosphatidyl ethanolamine, lysophosphatidyl ethanolamine, and branched fatty acid ester of hydroxy fatty acid.

4. A method for preparing a phospholipid-based surfactant, comprising:
a seed culture step of culturing a strain of skin flora with the accession number KCTC15741BP (Korea Research Institute of Bioscience and Biotechnology, Biological Resource Center) to prepare a seed culture;
a pre-culture step of culturing the seed culture in a medium containing vegetable oil to prepare a pre-culture;
a main fermentation step of adding the pre-culture to a medium containing vegetable oil to prepare a main fermented product;
a step of separating a precipitate from the fermented product; and
a step of extracting phospholipids from the precipitate.

5. The method for preparing a phospholipid-based surfactant according to claim 4, wherein the medium in the seed culture step contains casein, soybean, dextrose, sodium chloride and dipotassium phosphate.

6. The method for preparing a phospholipid-based surfactant according to claim 4, wherein the medium in the seed culture step contains 5 to 30 g/L of casein, 1 to 10 g/L of soybean, 1 to 6 g/L of dextrose, 1 to 10 g/L of sodium chloride, and 1 to 6 g/L of dipotassium phosphate.

7. The method for preparing a phospholipid-based surfactant according to claim 4, wherein the medium in the pre-culture step contains soybean, yeast extract, glycerin, potassium phosphate, dipotassium phosphate and vegetable oil.

8. The method for preparing a phospholipid-based surfactant according to claim 4, wherein the medium in the pre-culture step contains 0.01 to 3 g/L of soybean, 0.1 to 5 g/L of yeast extract, 0.1 to 5 g/L of glycerin, 1 to 15 g/L of potassium phosphate, 1 to 10 g/L of dipotassium phosphate, and 10 to 100 ml/L of vegetable oil.

9. The method for preparing a phospholipid-based surfactant according to claim 4, wherein the medium in the main fermentation step contains soybean, yeast extract, glycerin, potassium phosphate, dipotassium phosphate, ammonium sulfate, magnesium sulfate, potassium nitrate, calcium chloride, and vegetable oil.

10. The method for preparing a phospholipid-based surfactant according to claim 4, wherein the medium in the main fermentation step contains 0.01 to 3 g/L of soybean, 0.1 to 5 g/L of yeast extract, 0.01 to 5 g/L of glycerin, 1 to 10 g/L of potassium phosphate, 1 to 10 g/L of dipotassium phosphate, 0.1 to 5 g/L of ammonium sulfate, 0.01 to 5 g/L of magnesium sulfate, 0.01 to 3 g/L of potassium nitrate, 0.01 to 3 g/L of calcium chloride and 10 to 800 ml/L of vegetable oil.

11. The method for preparing a phospholipid-based surfactant according to claim 4, wherein
the seed culture step comprises culturing under aerobic conditions of 25 to 35° C.,
the pre-culture step comprises introducing 10 to 500 g/L of the seed culture and culturing under aerobic conditions of 25 to 35° C., and
the main fermentation step comprises introducing 10 to 500 g/L of the pre-culture and culturing under aerobic conditions of 25 to 35° C.

12. The method for preparing a phospholipid-based surfactant according to claim 4, the method comprises:
a step of subculturing to the pre-culture medium when the absorbance at a wavelength of 600 nm is 0.05 to 0.2 in the seed culture step;
a step of subculturing to the main fermentation when the absorbance at a wavelength of 600 nm is 0.2 to 1 in the pre-culture step; or
a step of completing the main fermentation at 60 to 150 hours after the start of the main fermentation.

13. The method for preparing a phospholipid-based surfactant according to claim 4, wherein the step of separating the precipitate comprises centrifuging the fermented product to recover the precipitate.

14. The method for preparing a phospholipid-based surfactant according to claim 4, wherein the step of extracting the phospholipids comprises
mixing the recovered precipitate and a solvent to obtain a filtrate; and
concentrating the filtrate.
